(19) United States
(12) Patent Application Publication
(10) Pub. No.: US 2016/0184351 A1
(43) Pub. Date: Jun. 30, 2016

(54) NEW COMPOSITION TO TREAT INFLAMMATION

(71) Applicant: IMMPERIODERM, Néville (FR)

(72) Inventor: Arnaud MAINNEMARE, Herborn (LU)

(21) Appl. No.: 14/909,598

(22) PCT Filed: Jul. 31, 2014
(86) PCT No.: PCT/EP2014/002098

§ 371 Date: Feb. 2, 2016

(30) Foreign Application Priority Data
Aug. 2, 2013 (FR) 13/01868

(51) Int. Cl. A61K 33/20; A61K 9/00; A61K 31/185

(52) U.S. Cl. CPC A61K 33/20; A61K 31/185; A61K 9/0063

(57) ABSTRACT

A composition for treating inflammation in a patient by administering within an exudate of the patient, which composition includes a concentration of less than or equal to 125 mM of one or more active ingredients salt for treating one or more symptoms of such inflammation, preferably less than or equal to 100 mM, and particularly preferably less than or equal to 75 mM.

This international patent application claims priority from French application FR 13/01868 filed Aug. 2, 2013, which is incorporated herein by way of reference.

The present invention is related to inflammation of the mucous membranes and relates more particularly to chronic and aggressive periodontitis with the provision of a specific composition for the treatment thereof.

With regard to the inflammatory process, it is a combination of reaction phenomena triggered in a living organism being attacked and implementing the immunological defence process. This “normal” process includes the local phenomena (which realises the study of a tissue fragment) and general phenomena (expressed clinically as fever and eventual altered condition of the biological inflammatory syndrome).

Now, this process is an omnitissular phenomenon that normally tends to reduce, eliminate and repair the effects of the attack. It ends with the repair of the lesion and cannot be undone in vascularised tissue.

Found at the origin of this process is the secretion of immune mediators which may be pro- or anti-inflammatories. These mediators can alter or maintain the inflammatory response. There are exogenous and endogenous pathogenic agents whose mode of action is not unequivocal. However, infections do constitute a small part of the inflammation caused. . . Among the exogenous factors responsible for inflammation, physical causes can be listed (trauma, heat, cold, ionising radiation, etc. . . . ), chemical causes (foreign bodies, caustics, toxics, etc. . . . ), the biochemical causes (allergens or any antigenic substances particularly food) or infectious agents, can act locally or remotely via toxin agents (microbes, viruses, parasites, fungi, etc). We can now lastly add to those endogenous elements with trophic causes (vascularisation or innervation disorders), degenerative lesions, metabolic disturbances (urea, gout . . . ), immune causes (auto -immunity, immune deficiency, or dysimmunity) or any lesions not based on inflammation such as tumours or atheroma.

This inflammatory process can be defined as a sequence of events, the main steps are:

1. Step of destructing and eliminating the attack causing inflammation.

2. Reversing the process with the restoration of tissue homeostasis, stopping the elimination phase and initiating the healing/repair phase.

3. Repair/healing phase.

The initial events are very difficult to grasp. Schematically, the contact between the pathogen and the system may trigger the following phenomena, which are not exclusive to each other : (i) Complete or partial elimination of pathogenic process by non-specific defence cells, NK cells, macrophages, mast cells, Paneth cells . . . which directly produce toxic proteins for the pathogen (eg: defensive Paneth cells, toxic protein granules of mast cells and NK, vasoactive substances (histamine, serotonin), . . . ); (ii) the pathogen captures and presents its antigens by a cell located at the boundary between the outside and the system. This boundary is situated at the epithelial level (digestive and bronchial systems) The cells involved may be epithelial cells, Langerhans cells, intraepithelial lymphocytes. During this interaction mediators (pro-inflammatory cytokines, inducible chemokines) are products that will firstly activate endothelial and mononuclear cells, on the other hand attract (chemotaxis) inflammatory cells to the location of the conflict.

Some cell damage is generated. They may be reversible, linked to metabolic disturbances, or irreversible alterations with nuclear and/or cytoplasmic.

In connection with the inflammation, it often appears that oedema is the result of an active phenomenon due to the passing from congestive vessels towards the middle interstitial a liquid close to the plasma. This passing is linked to increased hydrostatic pressure and especially increased permeability of the vascular wall of capillaries and venules. This oedema results in diluting the inflammatory source, limiting this source by a fibrin barrier (fibrinogen), to focus instead on the means of humoral defence (immunoglobulins, complement, lysozyme) and provide the chemical mediators and slow down the circulatory current by hemoconcentration, which promotes the subsequent phenomenon, leukocyte diapedesis. The liquid flow resulting from an inflammatory oedema in a cavity is known as exudate, rich in proteins, which opposes the transudate, poor in fibrin, of mechanical origin.

An exudate is an outpouring of serous fluid due to an increase of the fluid pressure associated locally with changes in the permeability of the resulting membrane inflammation.

In the case of inflammation affecting mucous membranes, the presence of the inflammatory exudate greatly complicates the treatment of inflammation due to the exudate volume and pressure operated on the surrounding tissue. In general, the latter, once formed, takes significant time to reabsorb and contributes to the setting up of inflammation, especially in the case of periodontitis.

The inventor has now shown evidence that the same management within a periodontal pocket containing a specific exudate, a composition comprising at least one active ingredient, salt, allows a rapid decrease to be obtained, in the volume of the exudate and especially the treatment of inflammation of the tissue at the origin of the production of this exudation.

The resulting first purpose of the invention supported a composition for treating inflammation in a patient by administering within an exudate of said patient, which composition comprises a concentration of less than or equal to 125 mM of one or more active ingredients salt for treating one or more symptoms of such inflammation, preferably less than or equal to 100 mM, and particularly preferably less than or equal to 75 mM.

Such subject is a mammal and more specifically human; wherein a subject is suffering from mucositis associated with an exudate, preferably with periodontitis.

To enable the treatment to be effective, it should also be a function of the active ingredients, the concentration of said one or more of active salt ingredients is greater than or equal to 5 mM, preferably greater than or equal to 10 mM, and the method particularly preferred greater than or equal to 20 mM.

By “salt” is meant a calcium or sodium salt, preferably sodium.

Therefore, the composition according to the invention will have a sodium composition less than or equal to 125 mM, preferably less than or equal to 100 mM, and the method particularly preferred less than or equal to 75 mM. Simultaneously, this composition will include a sodium composition greater than or equal to 5 mM, preferably greater than or equal to 10 mM and particularly preferably greater than or equal to 20 mM.

“Active ingredient” means a molecule that has a therapeutic effect and which has in this case an anti-inflammatory, healing and/or anti-infectious effect.

Standard examples of active ingredients that can be listed are the anti-inflammatories, immunoregulators, wound healing stimulators and/or tissue repair, antiseptics, or lastly antimicrobials.

Preferably one or more active ingredient salts are a mixture of (i) sodium hypochlorite (NaOCl) and (ii) sodium salt of N-chlorotaurine (NCT).

Always recommended, is that the sodium hypochlorite concentration is greater than or equal to 7 mM, preferably greater than or equal to 15 mM. Now, a concentration is chosen of sodium hypochlorite which is advantageously less than or equal to 85 mM, preferably less than or equal to 70 mM, and particularly preferably less than or equal to 60 mM.

Ideally, the sodium hypochlorite concentration is between 30 and 45 mM.

With regard to the sodium salt of N-chlorotaurine, its concentration is preferably greater than or equal to 0.5 mM, preferably greater than or equal to 10 mM. Now we choose a sodium salt concentration of N-chlorotaurine is less than or equal to 55 mM, preferably less than or equal to 40 mM.

Ideally, this sodium salt concentration of N-chlorotaurine is between 20 and 30 mM.

The composition of the invention accordingly may further comprise a pharmaceutically acceptable carrier.

The term “pharmaceutically acceptable” refers to molecular entities or compositions that are physiologically tolerable and do not typically produce an allergic reaction or similar unbearable reaction, such as intestinal upset or dizziness when administered to the patient. Preferred method: the term “pharmaceutically acceptable” as used herein means approved by a regulatory agency of a federal government or a state or listed in the US Pharmacopeia or other generally recognised pharmacopeia for use in animals, and more particularly in humans.

The term “carrier” refers to a diluent, adjuvant, excipient, or vehicle with which the compound according to the invention is administered. Such pharmaceutical carriers can be sterile liquids, such as water or oils, including those of petroleum, animal, vegetable or synthetic origin, and those such as peanut, soybean, mineral, or sesame oils. Water or any aqueous solution, saline or aqueous dextrose or glycerol are preferred methods used as carriers, particularly for injectable solutions. For example, the composition may comprise emulsions, microemulsions, oil in water emulsions, anhydrous lipids and water-in-oil emulsions, or other types of emulsions. Pharmaceutically acceptable carriers are described in the publication “REMINGTON'S Pharmaceutical Sciences” by EW Martin. The composition of the invention may further comprise one or more additives such as diluents, excipients, stabilisers and preservatives. Such additives are well known to the skilled person and are described in particular in “Ullmann's Encyclopedia of Industrial Chemistry, 6th Ed.” (Different publishers, from 1989-1998, Marcel Dekker); and in “Pharmaceutical Dosage Forms and Drug Delivery Systems” (Ansel et al.,1994, WILLIAMS & WILKINS).

Finally, the composition according to the invention may be administered one or more times.

In terms of the destination of the composition according to the invention, it is advantageous for treating periodontitis and passes through administration within the periodontal pocket.

In this context, the administration of the composition according to the invention can be done on a patient which is to be the object of a scaling/root planing.

Such administration can be carried out using medical devices such as those described in U.S. Pat. Nos. 4,685,596, CA 2,029,295, CA 2,005,514, U.S. Pat. No. 5,330,357, U.S. Pat. No. 4,973,250 or U.S. Pat. No. 4,950,163.

A second object of the invention concerns a method of treating inflammation in a patient, comprising the administering of a composition as previously described within an exudate of that patient.

Now this inflammation is advantageous to periodontitis and administration within the exudate is an administration within the periodontal pocket.

The following examples detail the invention with reference to various methods. No limitation of the invention must be considered in the light of these detailed examples. The invention includes all embodiments which would include details not explicitly mentioned in the following examples, but would be readily understandable to those skilled in the art.

A controlled prospective, randomised multicentric and unblinded phase I-II clinical study is realised with randomisation, which study includes the following 4 groups:
i. A “T” or control group, in which the patients with periodontitis were treated with the current standard treatment corresponding to a scaling/root planing without subgingival irrigation.
ii. A group named “Dₖ” in which patients are treated with a scaling/root planing with subgingival irrigation by a Dakin's solution diluted with a Na + concentration of 154 mM.
iii. A group named “S1-154” in which patients are treated with scaling/root planing with subgingival irrigation with a solution of sodium hypochlorite and sodium salt of N-chlorotaurine with an Na⁺ concentration of 154 mM.
iv. A group named “ irrigation S1-55” in which patients are treated with scaling/root planing with subgingival irrigation with a solution of sodium hypochlorite and sodium salt of N-chlorotaurine with an Na⁺ concentration of 55 mM.

In the study, the inclusion criteria include any consulting adult in one of the research centres if it has (i) at least 20 teeth, (ii) an advanced chronic periodontitis defined by periodontal sites with depth pocket sample of ≧6 mm and a clinical periodontal attachment loss ≧5 mm and (iii) affecting at least 30% of teeth, including at least 3 teeth with one or several radicles. Naturally, written and signed consent will be obtained prior to inclusion of the patient.

This study will not include pregnant women, those with valvular or severe heart disease, immunodeficients, drug abusers, subjects that have been treated in the last three months with antibiotic, anti-inflammatory, or immunomodulatory drugs.

Within the study, the principal criteria will be the reduction of periodontal attachment loss clinic at 70^{th} day compared to the loss before treatment. The secondary criteria will be, i)—for the systemic safety, methemoglobinemia and impaired creatinine and transaminases; ii)—for local effects, periodontal pocket depth, indicators of bleeding on probing and tooth mobility; and iii)—for the assessment of periodontal immune effects, the dosage in the periodontal crevicular fluid of cytokines IL-1β, TNF-α and IL-17.

Concerning the conduct of the study, patients included will be required to make six visits during the first two weeks and then two further visits during the following eight weeks. The initial consultation will include a detailed medical questionnaire, preoperative evaluation of oral pain, indicators of gingival bleeding, tooth mobility and a blood sample. Scaling/root planing with or without accompanying irrigation will be done to the group randomly. . . The clinical periodontal attachment level will be measured by electronic survey with constant force at D0 and then every four weeks from D14 to D70.

In terms of the statistical method, the total number of subjects included therein 188: with 47 patients in each of the four groups T, Dₖ, S1-55 and S1-154. The total duration of the study will be 30 months. We will test the benefit of irrigation with a solution of HS-CT compared to non-surgical treatment without irrigation (comparing two groups S1 versus T) in relation to non-surgical treatment with irrigation (comparison of two groups S1 versus group Dₖ), the specific effect of irrigation with Dakin (comparison of group Dₖ versus T) and finally the anti-exudate effect (comparison of group S1-55 versus S1-154). Being used for the comparison analysis of variance with Dunnett post-tests at α-risk of 0.05

It is expected to demonstrate a significant improvement in the periodontal healing (disappearance of periodontal pockets and optimal gain in clinical attachment) through the subgingival irrigation with a solution of sodium hypochlorite and sodium salt of N-chlorotaurine (HS-CT) with reduction of complaints in cases of advanced periodontitis. The right tolerance of HS-CT solutions for local use will also be analysed. Thus, periodontal care accessibility should be vastly improved and strengthened.

1. A composition for treating inflammation in a patient by administering within an exudate of said patient, which composition comprises a concentration of less than or equal to 125 mM of one or more active ingredients salt for treating one or more symptoms of such inflammation, preferably less than or equal to 100 mM, and particularly preferably less than or equal to 75 mM.

2. The composition according to claim 1, in which the salt or salts are of calcium or sodium, preferably sodium.

3. The composition according to claim 1, wherein the salt or salts are sodium salts with a sodium concentration less than or equal to 125 mM and greater than or equal to 5 mM.

4. The composition according to claim 1, wherein the composition is a mixture of (i) sodium hypochlorite (NaOCl) and (ii) sodium salt of N-chlorotaurine (NCT).

5. The composition according to claim 4, wherein the composition has a concentration of sodium hypochlorite greater than or equal to 7 mM, preferably greater than or equal to 15 mM.

6. The composition according to claim 5, wherein the composition has a concentration of sodium hypochlorite of between 30 and 45 mM.

7. The composition according to claim 4, wherein the composition has a concentration of sodium salt of N-chlorotaurine greater than or equal to 0.5 mM, preferably greater than or equal to 10 mM.

8. The composition according to claim 7, wherein the composition has a concentration of sodium salt of N-chlorotaurine between 20 and 30 mM.

9. The composition according to claim 1, wherein said composition is intended to treat periodontitis and the administration thereof is carried out within the periodontal pocket.

10. The composition according to claim 1, wherein the composition further comprises a pharmaceutically acceptable carrier.
